(19) United States
(12) Patent Application Publication
(10) Pub. No.: US 2026/0139216 A1
(43) Pub. Date: May 21, 2026

(54) Computer Enabled System and Method for Production of Cell Therapy Products

(71) Applicants: Rogelio Figueroa, Del Mar, CA (US); Erin Stephens, Del Mar, CA (US); Michael Benedict, Del Mar, CA (US); Arturo Goznalez, Del Mar, CA (US)

(72) Inventors: Rogelio Figueroa, Del Mar, CA (US); Erin Stephens, Del Mar, CA (US); Michael Benedict, Del Mar, CA (US); Arturo Goznalez, Del Mar, CA (US)

(21) Appl. No.: 19/199,307

(22) Filed: May 5, 2025

Related U.S. Application Data
(60) Provisional application No. 63/722,259, filed on Nov. 19, 2024.

(51) Int. Cl. C12M 1/36; C12M 1/00; C12M 1/107; C12M 1/34; C12M 3/00; C12M 3/04

(52) U.S. Cl. CPC C12M 41/48; C12M 23/36; C12M 23/38; C12M 23/48; C12M 27/10; C12M 29/24; C12M 41/12; C12M 41/32; C12M 41/34; C12M 41/36; C12M 41/40; C12M 41/42

(57) ABSTRACT

A system is provided for computer control of production of a cell therapy product in a fluid container. The system provides a container mount for a reactor or fluid container. Software running in electronic memory operates to provide motion to the fluid container which is required for a chosen processing regimen for the cell therapy product within the container. Sequential inspection for accurate ongoing cell processing is provided by digital image comparison of control process images to current digital images whereby the process automatically continues. Remedial actions can be initiated by the system where current imagery does not match control images.

This application claims priority to U.S. Provisional Patent Application Ser. No. 63/722,259 filed on Nov. 19, 2024.

### BACKGROUND OF THE INVENTION

### 1. Field of the Invention

The invention herein disclosed relates generally to the field of cell therapy. More particularly, it relates to autologous, allogeneic, and progenitor cell therapy wherein the harvested cells may be modified in an automated system, configured for engagement with a fluid container and subsequently reinfused into a patient as a treatment for illness.

### 2. Prior Art

In modern medicine, cell therapy is a treatment which involves processing cells to achieve an effect or change therein, and then injecting, grafting, or implanting viable cells from the process into a patient to achieve a medicinal effect. There are several types of cell therapy, including cellular adoptive immunotherapy, which involves isolating and reinfusing the immune cells of the patient, such as T-cells, to fight disease. This and other cell therapies are employed to treat patients for various illnesses, such as for treating cancer.

Consequently, advanced therapy manufacturing products, such as cell and gene therapies, represent a groundbreaking frontier in modern medicine, offering the potential to treat, or even cure, previously incurable diseases by harnessing and modifying biological cells. Such therapies promise transformative benefits for patients with conditions ranging from genetic disorders to cancer.

However, the successful realization of these benefits hinges on the ability to produce high-quality, consistent cellular products through advanced manufacturing processes. Therefore, the development of innovative manufacturing devices and systems is crucial to insure precise control, scalability, and reproducibility, ultimately enhancing the efficacy and safety of such systems and processes.

In the process of identifying and isolating desirable cells for such reinfusion to a patient, automated cell culture systems are widely employed. Such systems perform tasks, such as diluting samples, identifying desirable cells for infusion, growing cell cultures, and plating cultures.

However, the vast majority of such cell processing systems and components require constant adjustments and monitoring, and much of the processing must be done by trained human technicians during the entire process. Such limits the employment of such processes to labs and medical facilities having trained personnel, and also limits the availability of the benefits of these ground breaking health technologies.

With respect to the above, before explaining at least one preferred embodiment of the system herein involving the manipulation of genes, cells, or tissues in a safe manner so they may be administered to patients. It is to be understood that the system and device herein is not limited in its application to the details of employment, and to the arrangement of the components or the steps set forth in the following description or illustrated in the drawings. The various components, mechanical, and software-enabled methods and steps of the herein disclosed are capable of other embodiments, and of being practiced and carried out in various ways, all of which will be obvious to those skilled in the art once the information herein is reviewed.

Also, it is to be understood that the phraseology and terminology employed herein are for the purpose of description, and should not be regarded as limiting. As such, those skilled in the art will appreciate that the conception upon which this disclosure is based may readily be utilized as a basis for other other cell and medical processing systems and components. It is important, therefore, that the embodiments, objects and claims herein, be regarded as including such equivalent construction and methodology insofar as they do not depart from the spirit and scope of the present invention.

### SUMMARY OF THE INVENTION

The disclosed system herein provides an automated computer-controlled system configured for engagement of a conventional bioreactor, or fluid container, for medical cell bioprocessing, which in addition to having a small physical footprint, yields a state-of-the-art processing of the cells within the engaged fluid container, which is configured to significantly advance the field of advanced therapy manufactured products, such as gene therapy, cell therapy, and tissue engineering. The cell processing system herein is configured to control the environment, movement, temperature, and other variables for each type of cell processing being conducted in the engaged fluid container.

To control the movement, timing, and other required laboratory and processing aspects of the processing within a conventional fluid container operatively engaged with the system. Software operating to the task of performing each of the identified tasks, for each type of cell processing of the system herein may be employed to increase production and enhance the reliability of such processing. Such software may operate with artificial intelligence(AI) and learning ability for the control of the environmental and movement operations of the system. By AI is meant herein software operating in electronic memory to perform the stated individual, or plurality of, tasks, which may include algorithms, routines, learning code or algorithms, or other code, enabling the software to learn and better determine ongoing actions and timing of the different actions to better the outcome of the process.

The integration of software configured to the task of controlling multiple variables which affect the outcome of the bioprocess provides for automation of the cell processing system thereby providing for a significant reduction in labor and material costs and enhancement in outcomes. Such software-enabled actions also allow for simplified logistics, and a wider deployment of the system herein to smaller labs and processing facilities heretofore unable to handle the skilled labor and tasks of cell processing in a medically viable manner. Such, of course, significantly increases the availability of bioprocessing, and the related patient treatments available therewith to locales including hospitals, laboratories, or even homes by reducing requirements for material, equipment transportation, or any other associated delays.

The engageable interacting components and computer-assisted control of the system and components herein ensures the integrity and quality of cells during the manufacturing process, and the cells produced for medical use from the outcome of that process.

One preferred element to the cell processing system herein is the employment of a container base, which is configured to engage with and provide appropriate movement to a fluid container employed for holding cells in a processing solution. Conventionally, such fluid containers, known as bioreactors, are positioned in locations in processing labs where the required observation of the contents, and the movement and adjustments to the variables, which interact with the contents of the container during the cell processing regimen, is performed by lab technicians. Such requires trained technicians and is expensive, time consuming, and has an outcome directly proportional with the skill of the technicians interaction with the processing containers.

In the bioprocessing system herein, a container base is configured for operative engagement with a fluid container which is employed for holding cells within the liquid medium to be processed. By operative engagement herein is meant that fluid container removably couples at a lower end with the container base and removably couples at an upper end of the fluid container, with a rotatable top coupling which removably seals the container opening, wherein the fluid container may be agitated, rotated, or translated by movements of the container base while so engaged.

The fluid container of choice by the user having cells and liquid medium therein, is engaged with a container base component which is software controlled to impart the appropriate movement to the container base for the cell processing regimen chosen. The term regimen herein is used to describe cell processing of cells and media within a fluid container from a starting time to a finishing time wherein the fluid container with the processed cells is removed and the cells are used for the reason intended such as infusion within a patient. This term is for convenience, and should not be considered limiting, in that there appears no standard industry term to describe the term of the processing. This container base is as such, configured to operatively engage with the fluid container of the user, and thereafter provide precise adjustment of variables, which affect each individual bioprocessing regimen chosen.

Various types of cell processing regimen of cell cultures in such bioprocessing operations are affected by different environmental interactions. Such environmental variables include one, or a combination of, environmental variables from a group of environmental variables including:

Temperature communicated to the contents of the fluid container, carbon dioxide levels within the fluid container, oxygen levels within the fluid container, agitation speed of the fluid container and its contents, humidity within the fluid container, and an inclination (level or angle of incline) of the fluid container. The change of, or communication of, or non communication of each such environmental variables to the cells and media contents of the fluid container, has a known predictable effect upon the cell culture within the fluid container during the duration of cell processing.

In a fluid container, so employed, such cell processing primarily involves cell proliferation, cell differentiation, cell expansion, and cell harvesting of a specific type of cell, depending on the cell type being processed and desired outcome.

Cell proliferation is one of the most basic processes, where cells within the medium in the fluid container are provided with optimal conditions known to cause the cells to rapidly divide and increase in number. Cell proliferation processing is often used for large-scale cell production for therapeutic applications.

In cell differentiation processing, cells within the media within the fluid container are guided to develop into specific cell types with specialized functions. Cell differentiation is achieved by manipulating the culture medium in which the cells are mixed with specific growth factors and signaling molecules.

Cell expansion processing involves achieving a large-scale increase in one, or a specific, cell population within the media held by the fluid container. Such cell expansion processing is typically used to generate sufficient cells within the medium for transplantation, or further downstream processing.

A still further type of cell processing employing the fluid containers used in combination with the system herein, is that of cell harvesting. In the cell harvesting process specific cells within the fluid medium in the fluid container are identified and then carefully removed. Such can involve techniques like centrifugation, or filtration depending on the cell type and desired outcome.

In operation of the system herein, once the fluid container employed by the user is engaged with the container base, one or a plurality of video cameras capture constant or timed digital images of the cell and media contents within the fluid container. The captured digital images provide a current image of the cell and media contents at any given moment or time duration.

This captured current image is employable for comparison to a database of sequential captured images of cell and media contents, which have been determined to be in an optimum state at a respective time during the duration of the entire process of bioprocessing. Such bioprocessing for each type of cell processing, conventionally, has a time duration over which the cell and media within the fluid container are processed to reach a determined outcome. Conventionally, this processing is done by hand, and requires constant handling of the fluid container, and in many cases an opening of the container to take samples of the contents.

In the cell processing system herein, for each type of cell bioprocessing there may be stored in electronic memory, sequential control digital images which may be captured and stored to electronic memory. The sequentially captured control digital images depict a correct digital image of the media contents within a fluid container for a time during the specific individual bioprocess, or cell processing regimen, which has yielded proper or successful final outcomes for each cell processing process. The control digital images may be captured in a sequential fashion such that at specific time durations in the entire duration of the process, sequential control digital images are captured of a particular type of bioprocessing of cells, where the digital captured control images are deemed optimum or correct for that bioprocess at the captured time, during the entire time sequence of the entire bioprocess.

During the automated bioprocess provided by the system herein, digital cameras operate to capture digital images of the media contents such as the cells and surrounding liquid within the fluid container engaged to the container base of the system. The captured digital images of the fluid container contents may be captured at designated capture times, or time durations, such as sequentially at designated times over the entire processing time duration. These captured digital images from each of the capture times may be compared to the respective time-matching digital images in the library of control digital images for the process time correlating to the capture time of each captured digital image.

Where a digital image comparison shows that the media and cells in the captured digital image is substantially the same as that of the control digital image, then processing continues. Where a variance in the captured digital image from the control digital image is discerned, software operating to the task may either cause components of the system to take a known remedial action to change the environment for the contents of the fluid container, which is known to cause the contents to return to a proper configuration, or it may operate to notify a technician of the problem.

Both the control digital images stored in electronic memory, and the captured digital images at designated sequential times during the entire term of bioprocessing may be captured, and compared from multiple digital cameras and views. Such is preferred in the system herein to provide the most accurate digital review and outcome.

A top positioned digital camera may capture control digital images and process captured digital images from a digital camera at the upper end of the reactor container. A side-positioned digital camera may capture such control digital images and process captured digital images viewing the fluid container and contents from a side of the container substantially at a mid point thereof. Finally, a microscope digital camera may capture microscopic control digital images and process digital images for comparison using a digital camera with a microscopic lens.

Control digital images for each bioprocess at respective designated time durations may be captured by all three cameras for electronic storage and later compared with captured digital images from one, two, or all three digital cameras during the bioprocess at the designated capture times. The control digital images may be captured by the system provider using the system herein to operate a cell processing regimen wherein timed sequential control images are captured during a successful cell processing.

Image comparison software, operating to the task of comparing the control digital images sequentially taken and stored in electronic memory with the captured digital images at the proper times during processing, may monitor to discern multiple visually detectible attributes. As noted, the control digital images, and the captured digital images, may be from one or both digital cameras and/or the microscope which captures digital microscopic images.

By visually detectible attributes herein is meant to include one or a combination of visually detectable attributes from a group including:

Cell density where the digital imagery is examined for expected cell growth dynamics; cell clumping or cells having a proper homogeneous distribution; cell morphology via Microscopic Imaging of Cells to determine health and characteristics of cells; media color wherein the proper level of spent media is determined; media turbidity where software looks for cell concentration or presence of contaminants; cell viability percentage using infrared cameras, to determine if cell viability is substantially at an optimal range of more than 90%; and thermal imaging for temperature gradients in the fluid container. The cameras noted above can include thermal imaging ability or adjacent mounted thermal imaging cameras for such.

Where the digital image examination handled by comparison software of captured digital images to control digital images shows a substantial match of current visually discernable attributes to those in the control digital images for the matching time of capture, the software may signal the system to continue operations. Where differences between the visually discernable attributes of the captured digital image and the control digital image for the matching time of capture occur, the image examination software may take preprogrammed steps to alter the contents and interior environment of the fluid container to bring it back to nominal, or, it may take the step of informing a technician of a discerned problem between the two compared images.

In addition to the comparison of captured digital images during processing to the control digital images in electronic memory, electronic sensors may be positioned to take measurements of the contents of the environment in the fluid container.

Electronic sensors may monitor the environment of the fluid container for one, or a combination, of the following which are examples only and which environmental parameters or fluid container parameters will change, depending on the type of cell processing occurring:
Environmental Parameters:
Temperature (° C.) where a current preferred
range is 36.5° C.-37.5° C.
Humidity for an optimal Range of 85%-95% (in incubator)
CO Levels where a current optimal Range: 5%±0.5%
O2 Levels (%) Optimal Range: 18-21%
Pressure (kPa) Optimal Range: 101.3 kPa (atmospheric) or controlled variations for gas infusion;
Bioreactor & Liquid-Based Parameters:
Dissolved Oxygen such as an optimal Range: 175 M-204 M
Dissolved CO2 (ppm) such as an Optimal Range: 20-100 ppm
pH Levels such as in an Optimal Range: 7.0-7.4
Osmolality (mOsm/kg) such as an Optimal Range: 270-330
mOsm/kg
Glucose Concentration (g/L) such as Optimal Range: 1-5 g/L
Lactate Concentration (mM)such as an Optimal Range: <20 mM
Ammonia Concentration (mM)such as an Optimal Range: <5 mM.
Electronic sensors are available widely to monitor the above
   environmental and bioreactor liquid-based parameters and can be operatively placed upon a support adjacent to the position of the engaged fluid container, to sense the adjacent fluid container and send signals of current levels of such parameters to parameter software operating to the task of monitoring such parameters to match them to control parameters for each, captured at a point in time during the entire term of the bioprocess being performed.

If a match is discerned between a captured parameter and a control parameter in electronic memory for the point in time of the bioprocess, then the parameter software would allow the bioprocess to continue. Should a variance be discerned the parameter software will either initiate a system response to input changes in gas, air, temperature, or other noted parameters to the container, or will alert a technician of the determined variance.

Additionally, processing software operating to the task of movement control by controlling electric motors, which impart movement to the container once engaged to the container base component, may be employed. Such processing software can be customized to provide the specific different movements stored in electronic memory for the required sequential times during each cell processing procedure. The processing software may also be augmented with augmented movement software operating to the task of employing inputs from the image capturing software, and/or the parameter software to thereafter make changes in movements of the container by controlling movements of the electric motors moving the container base, in order to rectify some discerned problem from captured images or captured parameters.

Other processing modules may be included with the base processing module to further control the cell processing of the device and system herein. Such may include an environmental module such as a housing, to surround the precessing module having the container base configured for engagement to the user chosen fluid container, and control temperature and humidity surrounding it.

In all modes of the system and steps herein noted, the device and system provider and/or users thereof, may employ network accessible servers and/or computers having accessible electronic memory for storage and retrieval of the electronic database information relating to each monitoring and processing step in each software controlled process to the stated task, step, or requirement of the operation noted to be performed by the software. Software, running in electronic memory, will operate to perform each step, task, or calculation herein.

As to electronic memory or computer readable media for the system herein, any combination of one or more computer-usable or computer-readable media, be it transitory or non-transitory, may be employed for operation of the software operating to the stated tasks and any software assessment system herein. Such, for example, and in no way limiting, can include computer-readable media and may include one or more of a portable computer diskette, a hard disk, a random access memory device, a read-only memory device, an erasable programmable read-only memory (EPROM or Flash memory) device, a portable compact disc read-only memory device, an optical storage device, and other electronic memory magnetic storage devices. Software or computer program code for carrying out the individual and sequential operations and airflow assessments of the present invention may be written in any combination of one or more programming languages.

The steps or method of operation and/or execution of the various modes and tasks of the system herein may be illustrated as blocks or steps in the drawings, which may represent one or more sequences in the operation of the steps and assessments in the system herein. These operations or steps can be implemented using hardware, software operating to process input data to accomplish the stated task or step, or a combination thereof.

With regard to software operating to a task, steps, or assessments indicated in the system herein, such represents computer-executable instructions stored upon one or more transitory or non-transitory computer-readable storage media, which, when executed by one or a plurality of processors, will operate to perform the recited task, assessment, operation or step. Computer-executable instructions, in general, include routines, programs, algorithms, data structures, and the like, which are configured to perform particular functions or to implement particular abstract data types or steps noted.

It should be noted that the sequence in which the steps of the system herein are described or depicted are not intended to be construed as a limitation. It should be understood that any number of the described or designated steps in any task or process noted herein can be combined in any order and/or in parallel to implement the described and depicted assessments and processes and tasks. In some modes of the system herein, one or more steps can be rearranged or omitted entirely. Still further, the software-enabled steps in the system herein can be combined in whole, or in part, with each other or with other steps or methods.

With respect to the above description, before explaining at least one preferred embodiment of the components and computer enabled system and method for production of cell therapy products, it is to be understood that the disclosed invention is not limited in its application to the details of operation nor the arrangement of the components or the steps set forth in the following description or illustrations in the drawings. The various methods of implementation and operation of the device system and method herein are capable of other embodiments and of being practiced and carried out in various ways, which will be obvious to those skilled in the art once they review this disclosure. Also, it is to be understood that the phraseology and terminology employed herein are for the purpose of description, and should not be regarded as limiting.

Therefore, those skilled in the art will appreciate that the conception upon which this disclosure is based may readily be utilized as a basis for the designing of other devices, methods and systems for the production of cell therapy products. Therefore, that the objects and claims herein should be regarded as including such equivalent devices, components, construction, steps, and methodology insofar as they do not depart from the spirit and scope of the present invention.

It is an object of this invention to provide a closed system wherein a fluid container operatively engaged with a base may remain closed during the entire duration of cell processing therein.

It is a further object of this invention, to provide such a closed cell processing system, wherein the progress of the cell processing within the engaged fluid container is monitored using digital imaging.

It is an object of this invention to provide a simplified widely employable device and system for processing the manipulation of genes, cells, or tissues within the fluid containers for administration to patients.

It is a further object of this invention to provide such a manipulation system, which is configured with a container base adapted for engagement with a fluid container having cells and media therein, which is employable in labs on a much wider scale than existing systems.

It is yet another object of this invention to provide such a cell processing system wherein progress of the cell processing at sequential times during the entire process is determinable by captured digital imaging compared to stored nominal control digital images and automatic abutments to environmental factors are initiated when captured images show a variance from nominal.

These, together with other objects and advantages, which will become subsequently apparent, reside in the details of the construction and operation of disclosed herein for a computer enabled device and system for production of cell therapy products for administration to patients herein as more fully hereinafter described and claimed, reference being had to the accompanying drawings forming a part thereof, wherein like numerals refer to like parts throughout.

Further objectives of this invention will be ascertained by those skilled in the art as brought out in the following part of the specification wherein detailed description is for the purpose of fully disclosing the invention without placing limitations thereon.

### BRIEF DESCRIPTION OF DRAWING FIGURES

The accompanying drawings, which are incorporated herein and form a part of the specification, illustrate some, but not the only or exclusive, examples of embodiments and/or steps of the device, methods, and systems for the production of cell therapy products herein. It is intended that the embodiments and figures disclosed herein are to be considered illustrative of preferred modes of the system rather than limiting.

In the drawings:

FIG. 1 is a depiction of the cell processing device of the system herein shown having a container base configured for operative engagement with a fluid container, to provide optical observation, movement, and processing variables to the fluid container.

FIG. 2 shows a platform on which the container mount configured for engagement to a fluid container is positioned above and showing an adjacent support for optical sensors, illumination, and digital camera positioning thereon.

FIG. 3 shows a sectional view through an engaged fluid container operatively positioned on an electric motor which is coupled to the container mount of the system herein, and showing gas and fluid engagements communicating environmental variables to the interior of the container.

FIG. 4 shows a sectional view through an engaged fluid container operatively positioned on the container mount of the system herein showing the view angle of an upper digital camera.

FIG. 5 shows a sectional view through an engaged fluid container operatively positioned on the container mount of the system herein, showing the view angle of a centrally positioned digital camera.

FIG. 6 shows a sectional view through an engaged fluid container operatively positioned on the container mount of the system herein showing the view angle of a digital microscope.

FIG. 7 depicts the forward, rearward, side to side, orbital, and spinning, or centrifugal, movements impartable to the container base and any container thereon by electric motors.

FIG. 8 depicts the container mount configured for operative engagement with a fluid container, which is positionable on a support surface such as in FIG. 7 and is moveable by electric motors.

FIG. 9 depicts a simple flow chart of the computer and software-enabled system and method herein for cell bioprocessing.

FIG. 10 is a graphic depiction of a video display, which may be employed to allow users to choose a processing regimen and allow the system to handle the entire process.

### DETAILED DESCRIPTION OF THE PREFERRED EMBODIMENTS OF THE INVENTION

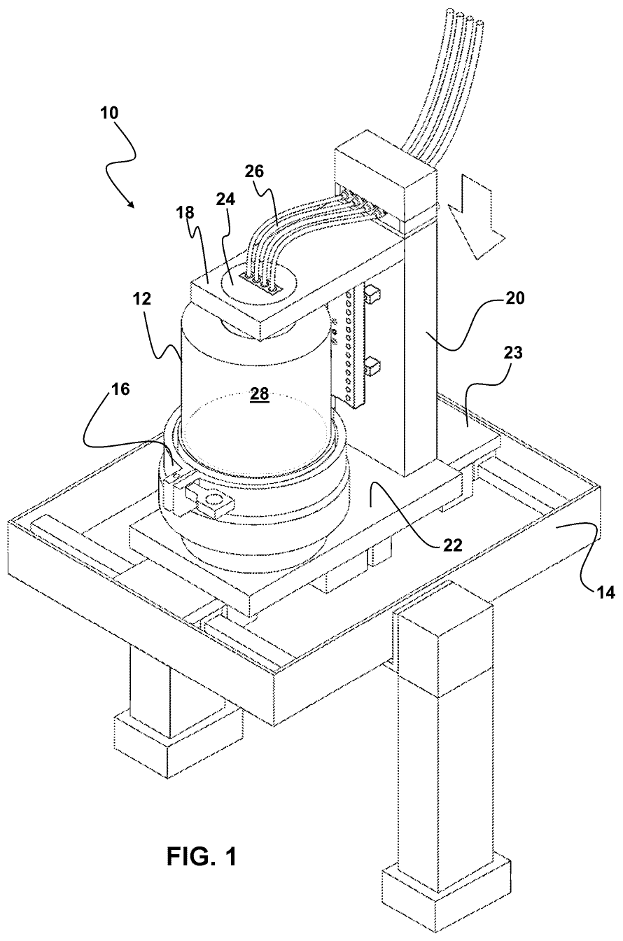

Referring now to the device and method and system 10 herein, shown in simple format by the depictions of FIGS. 1-8, as noted above, the system 10 may be modular in that one or more modules may be engaged using conduits, and the like, to achieve the desired cell processing. Software operating to the tasks of performing each noted step or task in the system 10 for medical cell bioprocessing may be employed to control one or all processes and tasks.

Shown in FIG. 1, is a simple depiction of the cell processing device 10 herein shown with multiple engageable components in a stacked configuration atop a support surface. As shown, the fluid container 12 is removably engaged with a reactor component or container base 16.

A top mount 18 extends from an engagement at one end with a support 20. The support 20 is coupled to a platform 22 operatively positioned on the support surface 14.

A tube mount 24 may be rotationally coupled with the top mount 18 to allow for rotational movement of the underlying fluid container 12 during bioprocessing. The tube mount 24 is configured to engage with a plurality of overhead tubes 26, each of which provides an interior passage for communicating gas and liquid such as mixtures of oxygen and carbon dioxide into and out of the interior chamber 28 of the fluid container at determined pressures and temperatures to adjust such within the interior chamber 28 during bioprocessing.

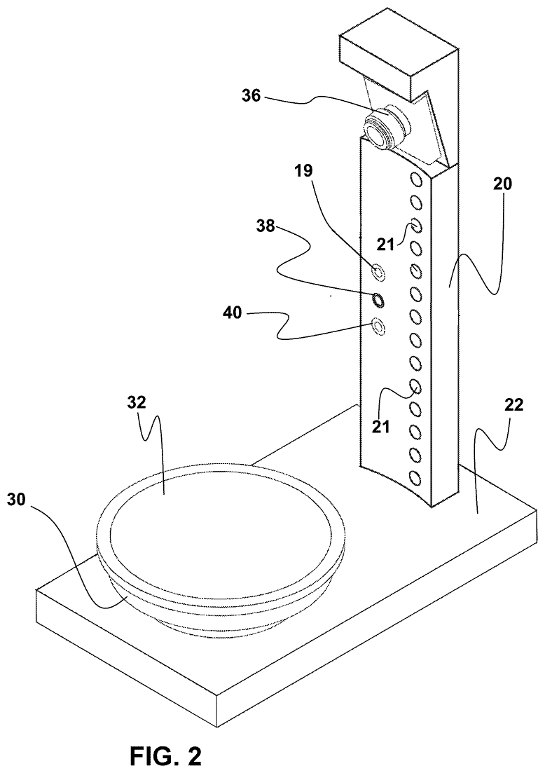

As shown in FIG. 2, the container base 16 as in FIG. 1, is coupled atop a spin platform 30 which is rotationally coupled to the platform 22. The spin platform 30 allows for rotation of the container base 16 and to any fluid container 12 mounted therein as the platform 30 is moved by an electric motor 57 as noted below. In some modes of the device 10 a central area 32 of a stationary imaging member may be configured as a light-emitting component such as a circular planar LED to provide illumination from below the fluid container.

Shown engaged with the support 20 in FIG. 2, is a overhead digital camera 36, which is positioned for a downward view of the interior chamber 28 of the fluid container 12. Also engaged to the support is a central digital camera 38 for capturing a view of a central area of the interior chamber 28 during processing. Additionally depicted is a digital microscope camera 40, which is configured for capturing microscopic images of portions of the contents of the interior chamber 28. Further, positionable upon the support are optical sensors 19 for environmental factors within the interior chamber 28 light emitters 41 for illumination.

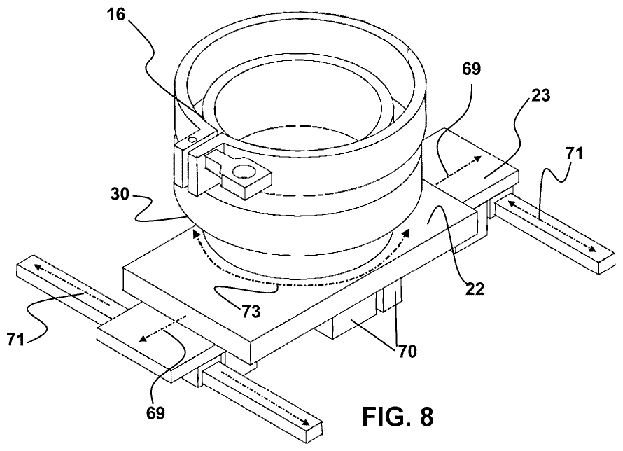

The platform 22 is shown in FIG. 8 in a translatable coupling to a mount 23 whereby the platform may translate in four directions using electric motors and the container base 16 will also rotate atop the platform 22 using electric motors to power the rotation.

Additionally depicted in FIG. 2 are a plurality of magnets 21 positioned on the support 20 on a side thereof facing the user fluid container 12 once operatively engaged to the container base 16. The magnets 21 be they permanent or electromagnets are employable for magnetic bead-based extraction of cells. Such magnetic bead-based extraction is well known and is employed during the cell processing regimen to purify biomolecules like DNA, RNA, or proteins depending on the cell processing regimen chosen by the user. For example, such magnetic beads may be coated with specific binding molecules where the biomolecule of interest to the user binds to the beads in a solution. The magnetic field is then applied to separate the beads (and the bound biomolecule) from the rest of the solution within the user fluid container 12. The purified biomolecule can then be eluded from the beads. In the system provided by the device 10 herein, the magnets 21 will be operated by the system and software operating to control them for a cell processing regimen chosen by a user which requires the magnetic beads and magnet 21 attraction for that portion of the processing.

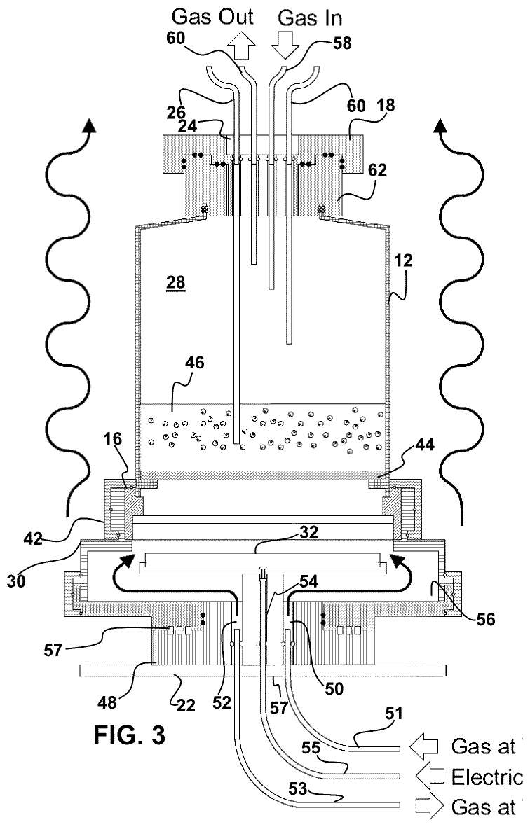

FIG. 3 shows a sectional view through the fluid container 12 for which the system herein is adapted to engage and container base 16 therefore. The fluid container 12 of the user is removably coupled to the container base 16 such as with reactor brackets 42. At a lower end of a conventional fluid container 12 is normally positioned a reactor membrane 44 which is configured to pass gas molecules therethrough to the interior chamber 28, but which prevents fluid 46 within the interior chamber 28 from flowing out. Such membranes are well known. A base mount 48 is operatively connected to the container base 16 and coupled with the platform 22. A first gas passage 52 engages with a first gas tube 53 to communicate gas into the interior chamber 28. The gas input from the gas input tube 53 follows a gas pathway through a gas chamber 56 and through the reactor membrane 44 into the interior chamber 28.

Gas may be drawn from the interior chamber 28 by passing through the reactor membrane 44 and through the gas chamber 56 to the gas exhaust tube 53. The gas such as oxygen or carbon dioxide may be added or removed from the interior chamber 28 during the processing operation as needed and as determined by the software monitoring the processing. The temperature of the gas in the gas input tube 51 may be adjusted to thereby adjust the temperature in the interior chamber 28. Also shown is an electric passage 54 for communication of electricity from a wire 55 to an electric motor 57 to power spinning of the container base 16 and any fluid container 12 coupled thereto. The wire may also provided power for any light emitting component such as an LED.

An upper gas input tube 58 and upper gas outlet tube 60 is shown communicating through the top mount 18 and through a rotating top coupling 62, which seals the opening in the opening at the upper end of the fluid container 12 and allows for movement of the engaged fluid container. The top coupling 62 is in a rotational coupling with the top mount 18 such that the container 12 can rotate with it and stay sealed.

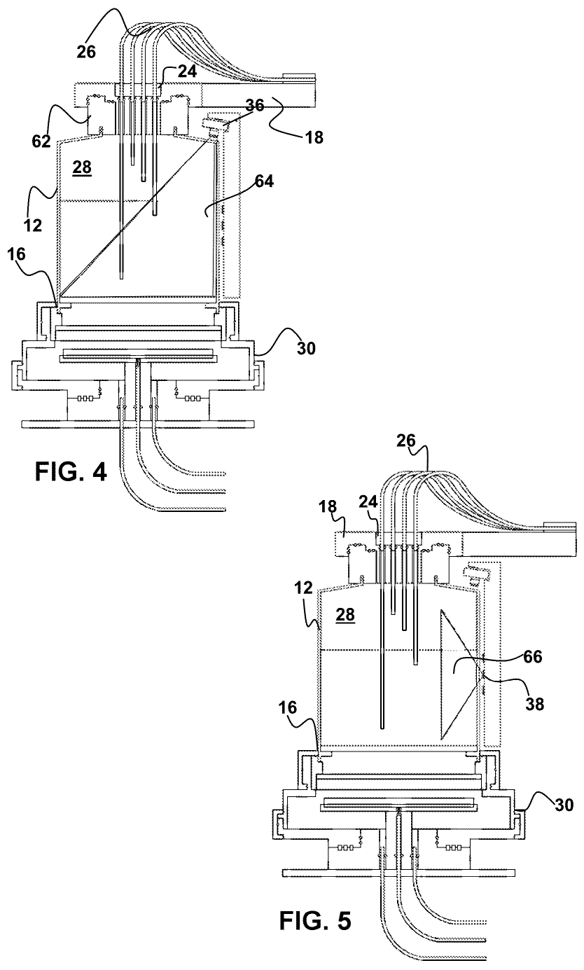

Shown in FIG. 4 is a sectional view through the fluid container 12 and container base 16 of FIG. 1. FIG. 4 depicts the view angle of an upper digital camera 36 which allows for digital images of the contents of the interior chamber 28.

FIG. 5 is a sectional view through the fluid container 12 and container mount container base 16 of FIG. 1, and showing the central view angle 66 of a centrally positioned digital camera 38.

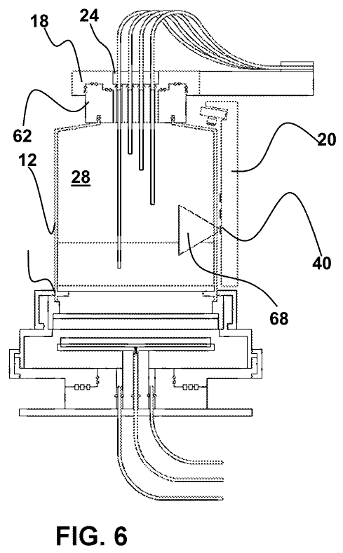

FIG. 6 depicts another sectional view through the fluid container 12 and container base 16 of FIG. 1 and showing the microscope view angle 68 of a digital microscope 40.

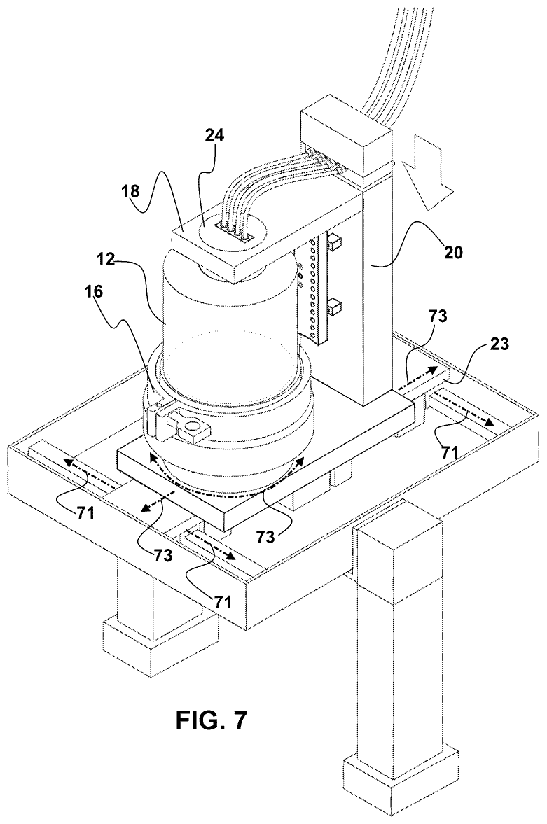

FIGS. 7-8 depict the movements imparted to the base and the container thereon by electric motors and/or actuators which enable precise mixing, swirling, or agitation as required for various processing steps in the cell processing regimen where each may be different and at different sequential times during the entire processing regimen.

FIG. 8 as noted shows the translating coupling of the platform 22 to a mount 23 wherein a linear translation along a first axis 69 is powered by electric motors 70 as well translation along a second axis 71. A linear electric motor or geared connection of the electric motors 70 may provide the translating movement along the second axis 71. As noted, rotational movement 73 of the container base 16 and any fluid container 12 operatively engaged therein, is powered by an electric motor 57 (FIG. 3). As such, during the ongoing term of cell processing within an interior chamber 28 of an operatively engaged fluid container 12, translating movement 73 as well as rotational movement, such as for centrifugal action, can be communicated to the tube mount 24 and thereon to the fluid container 12 and fluid 46 therein.

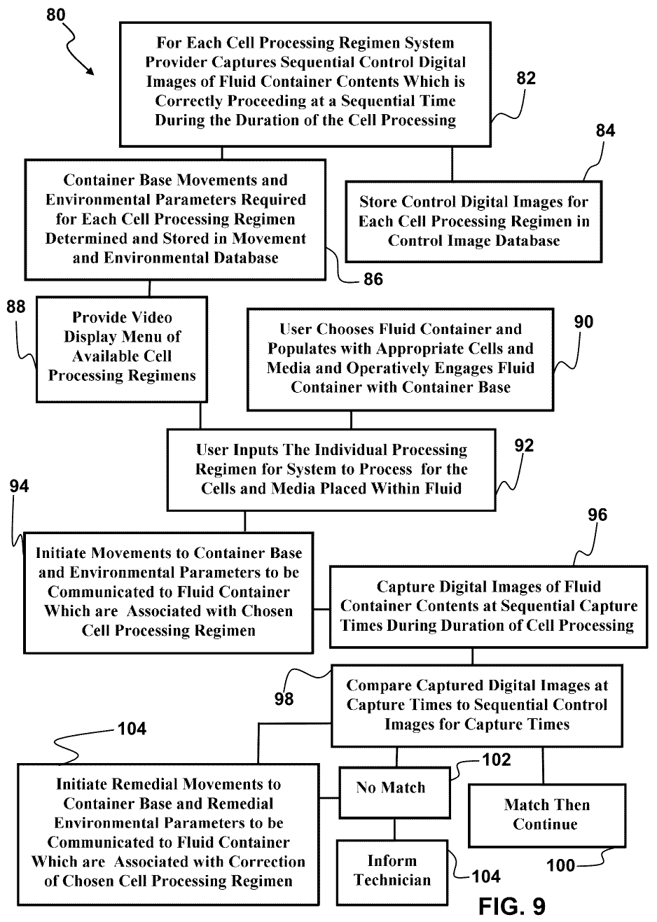

FIG. 9 depicts a simple flow chart of the computer and software-enabled method herein for cell bioprocessing. The steps shown are a simple depiction of the control and operation of the method 80 herein, which is controlled by software operating to the task, or tasks, required for each step which runs in electronic memory of a computer which in operative communication with all components herein as required to operate and monitor the system for each cell processing regimen for a user engaging a fluid container 12 with the mount 16 herein to allow the system 10 to operate the components in the method 80 herein to provide total control over the cell processing regimen for each user.

In providing the method 80 for operating and controlling cell processing for each cell processing regimen, the system provider will, for each respective cell processing regimen, the system provider will capture and maintain in a control digital image database, control digital images of the fluid container contents 82. The sequential control digital images, which are sequentially taken and used for comparison, depict a respective cell processing regimen, which is correctly proceeding at sequential respective comparison times during each cell processing regimen, during the total duration of the respective cell processing regimen. Such sequential control digital images are captured for each successful cell processing regimen where the cells and media within a fluid container yield a successful outcome of useable cells at the end of the regimen, and are stored in an electronic database of sequential digital control images for each respective processing regimen 84.

For each such cell processing regimen where the user places cells within a fluid container 12 for processing, there is a requirement, over the duration of the cell processing regimen, to move the container 12, and thus move the cells and media within it. In the method 80 herein, the required container base 16 movements to move the contents, and the environmental parameters required for each cell processing regimen for the duration of such, are determined and stored in a movement and environmental database 86. Such environmental parameters are noted above to include one or a combination of environmental parameters from a group including, temperature, humidity, C02 levels, 02 levels, atomospheric pressure, along with Bioreactor & Liquid-Based Parameters of dissolved oxygen dissolved CO2, pH Levels, Osmolality, glucose concentration, lactate concentration, and ammonia concentration. Thus the movements required of the container 12, which will be provided by the base 16 will be controlled by software operating to that task for the duration of the processing regimen. Based on inputs from sensors and images from the digital imaging components, the other environmental parameters may also be adjusted.

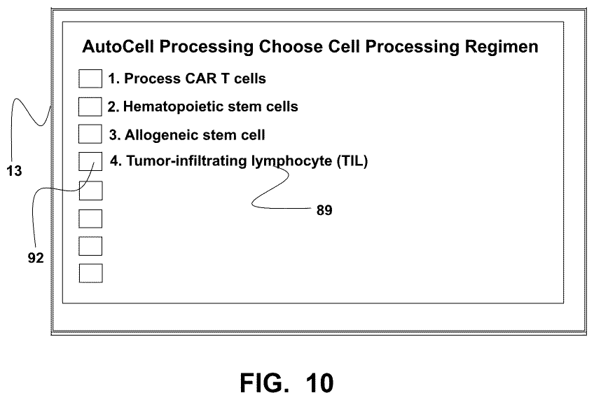

The system provider may provide a video display 13 (FIG. 10) or other menu 88 type means for the user to input the individual processing regimen 89, from those available. The system 10 will then initiate the chosen individual cell processing regimen 89 for the cells and liquid media placed within the user-engaged fluid container 12.

Using the system herein, the user will choose a fluid container 88 from the system provider or their own stock thereof, which is configured to operatively engage with the base 16. The user will deposit the liquid media and cells into a fluid container 12 and engage it with the container base 16.

For the cells and media the user deposited into the container which they operatively engage with the base, the user may designate 92 the individual cell processing regimen for the system 80 to perform upon the operatively engaged fluid container having the user-deposited cells and media therein.

Software operating to initiate the designated cell processing regimen 92 will choose and initiate any and all movements 94 to the container base and will choose and initiate the environmental parameters to be communicated to fluid container 12, using the gas input tubes and gas output tubes noted above, which are and have been respectively associated 86 with chosen cell processing regimen 92 which are stored in an electronic database in electronic memory. There is an infinite number of processing regimens 92 for choices of the user which the system may automatically process so long as the duration and environmental parameters and required movements during the duration of the chosen processing regimen are stored in electronic memory as related to each respective individual processing regimen offered on the video display 13 or other means for allowing the user to choose their desired processing regimen.

As the system operates to process the user chosen contents of the fluid container in the user chosen processing regimen 92, the system 80 will operate to at different times sequentially during the entire duration of the processing regimen 92, to capture digital images 96 at sequential capture times of the contents of the fluid container.

The captured digital images 96 at the capture times may be compared 98 to the stored control digital images 82 held in the database in electronic memory as associated with the chosen processing regimen 92 for the sequential time of the control digital images 82 which substantially matches the time of the captured digital image 98.

Where comparison software operating to compare the captured digital image to the stored control digital images determines a substantial match, to the stored digital image for the time during the chosen the duration of the cell processing regimen 100, then the system will continue to process the contents of the fluid container for the user chosen processing regimen 92.

Where comparison software operating to compare the captured digital image to the stored control digital images determines no match, to the stored digital image for the time during the chosen the duration of the cell processing regimen, the system may employ software operating to the task of identifying a mismatch, and then initiating remedial movements and initiating changes to environmental parameters, which have been pre-associated to correcting the identified mismatch 104. Such remedial movements and changes to environmental parameters may be determined and associated with identified mismatches, and stored in electronic memory for use by the comparison software.

Once the remedial movements and remedial environmental parameter changes have been communicated to the fluid container, software operating to make a comparison of newly captured digital images to the control digital images 98 will determine if there is a match to of the captured digital images. If a match is determined 100, processing will continue. If no match 102 is determined, the system may initiate the remedial movements and environmental parameters again, 104, or inform a technician 106 of the mismatch.

The software controlled system above enables the processing of the user chosen contents of cells and liquid media for the entire duration of the chosen processing regimen 92, without the need for the container to be opened. Further, as noted it eliminates processing errors frequently caused by inattention of technicians, and provides a much more standardized outcome of cell processing for each of the available cell processing regimens provided by software operating to the task of providing such.

While all of the fundamental characteristics and features of the device, methods, and systems for the production of cell therapy products have been shown and described herein, with reference to particular embodiments thereof, a latitude of modification, various changes, and substitutions are intended in the foregoing disclosure and it will be apparent that in some instances, some features or steps of the disclosed system may be employed without a corresponding use of other features without departing from the scope of the invention as set forth. It should also be understood that various equivalent substitutions, modifications, and variations, may be made by those skilled in the art without departing from the spirit or scope of the invention. Consequently, all such modifications and variations and substitutions are considered included within the scope of the invention herein disclosed.

What is claimed is:

1. A computer enabled system for processing of cell therapy products for a duration of a cell processing regimen, comprising:
a container base configured for coupling with a fluid container;
said container base rotationally coupled to a platform;
a motor for rotating said container base;
a computer processor, and
a non-transitory, computer readable medium communicably coupled to the processor and storing processing instructions that, when executed by the processor, cause the processor to perform operations comprising:
controlling said motor to impart rotational movement to said container base and any fluid container coupled thereto in rotational movements required for cell processing for a duration of time of a chosen processing regimen.

2. The computer enabled system for processing of cell therapy products for a duration of a cell processing regimen of claim 1, comprising:
said platform slidingly coupled to a support surface;
a translating motor for translating said support surface along at least one axis;
said processing instructions additionally controlling said translating motor to impart translating movement to said platform and to said container base and any said fluid container coupled thereto, to thereby communicate translating movements required for cell processing for a duration of time of a processing regimen.

3. The computer enabled system for processing of cell therapy products for a duration of a cell processing regimen of claim 1 additionally comprising:
a top coupling for positioning on an opening at an upper end of said fluid container; and
said top coupling in a rotational coupling with a top mount whereby said top coupling rotates in said rotational coupling as said container base rotates said fluid container.

4. The computer enabled system for processing of cell therapy products for a duration of a cell processing regimen of claim 2 additionally comprising:
a top coupling for positioning on an opening at an upper end of said fluid container; and
said top coupling in a rotational coupling with a top mount;
said top mount extending from a connection of a first end thereof, to an upper portion of a support; and
said support engaged at a first end to said support surface; and extending from said first end to said upper portion.

5. The computer enabled system for processing of cell therapy products for a duration of a cell processing regimen of claim 1 additionally comprising:
a support extending from said platform;
a first digital camera coupled to said support;
said first digital camera for taking first captured digital images of cells and fluid media positioned within an interior of said fluid container; and
said processing instructions operating additionally to compare said first captured digital images to control images stored in electronic memory for a match therebetween, and continue controlling said motor to impart rotational movement to said container base for said duration of time of said processing regimen only upon discerning said match.

6. The computer enabled system for processing of cell therapy products for a duration of a cell processing regimen of claim 2 additionally comprising:
a support extending from said platform;
a first digital camera coupled to said support;
said first digital camera for taking first captured digital images of cells and fluid media positioned within an interior of said fluid container; and
said processing instructions operating additionally to compare said first captured digital images to control images stored in electronic memory for a match therebetween, and continue controlling said motor to impart rotational movement to said container base and controlling said translating motor to impart translating movement to said platform, for said duration of time of said processing regimen, only upon discerning said match.

7. The computer enabled system for processing of cell therapy products for a duration of a cell processing regimen of claim 5 additionally comprising:
a second digital camera coupled to said support;
said second digital camera being a digital microscope for taking microscopic captured digital images of said cells and said fluid media positioned within said interior of said fluid container;
said processing instructions operating additionally to compare said microscopic captured digital images to control microscopic images stored in electronic memory for a microscopic match therebetween and continue controlling said motor to impart said rotational movement to said container base for said duration of time of said processing regimen upon discerning said microscopic match.

8. The computer enabled system for processing of cell therapy products for a duration of a cell processing regimen of claim 6 additionally comprising:
a second digital camera coupled to said support;
said second digital camera being a digital microscope for taking microscopic captured digital images of said cells and said fluid media positioned within said interior of said fluid container;
said processing instructions operating additionally to compare said microscopic captured digital images to said control images stored in electronic memory for a match therebetween and continue controlling said motor to impart said rotational movement to said container base and controlling said translating motor to impart said translating movement to said platform, for said duration of time of said processing regimen, upon discerning said microscopic match.

9. The computer enabled system for processing of cell therapy products for a duration of a cell processing regimen of claim 7 additionally comprising:
a third digital camera coupled to said support;
said third digital camera for taking third captured digital images of cells and fluid media positioned within said interior of said fluid container; and
said processing instructions operating additionally to compare said third captured digital images to third control images stored in electronic memory for third match therebetween, and continue controlling said motor to impart said rotational movement to said container base and controlling said translating motor to impart said translating movement to said platform, for said duration of time of said processing regimen, upon discerning said third match.

10. The computer enabled system for processing of cell therapy products for a duration of a cell processing regimen of claim 8 additionally comprising:
a third digital camera coupled to said support;
said third digital camera for taking third captured digital images of cells and fluid media positioned within said interior of said fluid container; and
said processing instructions operating additionally to compare said third captured digital images to third control images stored in electronic memory for third match therebetween, and continue controlling said motor to impart said rotational movement to said container base and controlling said translating motor to impart said translating movement to said platform, for said duration of time of said processing regimen, upon discerning said third match.

11. The computer enabled system for processing of cell therapy products for a duration of a cell processing regimen of claim 5 additionally comprising:
a top coupling for positioning on an opening at an upper end of said fluid container;
said top coupling in a rotational coupling with a top mount; and
said top mount extending from a connection of a first end thereof, to an upper portion of said support;
an upper gas inlet tube communicating through said top coupling;
an upper gas outlet tube communicating through said top coupling;
whereby said top coupling rotates in said rotational coupling as said container base rotates said fluid container and said upper gas inlet tube said upper gas outlet tube have portions thereof positioned within said interior of said fluid container.

12. The computer enabled system for processing of cell therapy products for a duration of a cell processing regimen of claim 6 additionally comprising:
a top coupling for positioning on an opening at an upper end of said fluid container;
said top coupling in a rotational coupling with a top mount; and
said top mount extending from a connection of a first end thereof, to an upper portion of said support;
an upper gas inlet tube communicating through said top coupling;
an upper gas outlet tube communicating through said top coupling;
whereby said top coupling rotates in said rotational coupling as said container base rotates said fluid container and said upper gas inlet tube said upper gas outlet tube have portions thereof positioned within said interior of said fluid container.

13. The computer enabled system for processing of cell therapy products for a duration of a cell processing regimen of claim 7 additionally comprising:
a top coupling for positioning on an opening at an upper end of said fluid container;
said top coupling in a rotational coupling with a top mount; and
said top mount extending from a connection of a first end thereof, to an upper portion of said support;
an upper gas inlet tube communicating into an interior of said fluid container through said top coupling;
an upper gas outlet tube communicating through said top coupling;
whereby said top coupling rotates in said rotational coupling as said container base rotates said fluid container.

14. The computer enabled system for processing of cell therapy products for a duration of a cell processing regimen of claim 11 additionally comprising:
said base rotationally coupled to a central portion thereof which is coupled to said platform;
a gas chamber positioned within said base;
a lower gas inlet tube communicating through said central portion of said base and into said gas chamber; and
a lower gas outlet tube communicating through said central portion of said base and into said gas chamber.

15. The computer enabled system for processing of cell therapy products for a duration of a cell processing regimen of claim 12 additionally comprising:
said base rotationally coupled to a central portion thereof which is coupled to said platform;
a gas chamber positioned within said base;
a lower gas inlet tube communicating through said central portion of said base and into said gas chamber; and
a lower gas outlet tube communicating through said central portion of said base and into said gas chamber.

16. A computer enabled system for processing of cell therapy products for a duration of a cell processing regimen, comprising:
a container base configured for coupling with a fluid container having an interior cavity for processing a cell therapy products therein in a processing regimen;
said container base rotationally coupled to a platform;
a motor for rotating said container base;
at least one camera positioned adjacent said platform in position for capturing current digital images of said cell therapy products within said interior cavity;
a computer processor, and
a non-transitory, computer readable medium communicably coupled to the processor and storing processing instructions that, when executed by the processor, cause the processor to perform operations comprising:
providing a listing of a plurality of different individual processing regimens having a time duration which are available for a user to designate a chosen processing regimen;
forming a control database of control digital images of cell therapy products taken at control sequential times during said time duration for each of said plurality of different processing regimens;
allowing a user to designate a said chosen processing regimen;
controlling said motor to impart rotational movement to said container base and any fluid container coupled thereto in rotational movements required for the chosen cell processing regimen for the chosen cell processing regimen;
actuating said camera to capture current digital images of said cell therapy products within said interior cavity, at said control sequential times;
comparing said current digital images to said control digital images; and
where a match is determined between said current digital image and said control digital image, allowing said chosen cell processing regimen to continue.

17. A system for controlling production of a cell therapy product in a fluid container, having electronic data sensors positioned for continued inspection of said cell therapy product, comprising:
a processor, and
a non-transitory, computer readable medium communicably coupled to the processor and storing instructions that, when executed by the processor, cause the processor to perform operations comprising:
determining a time duration for a processing regimen for an individual cell therapy product within an interior of a fluid container;
determining a number of sequential time points during said time duration of said processing regimen, for a respective inspection of said cell therapy product, while positioned within said interior of said fluid container;
from a group of environmental parameters including one or a combination of temperature, humidity, C02 levels, 02 levels, atomospheric pressure, dissolved oxygen, dissolved CO2, pH Levels, Osmolality, glucose concentration, lactate concentration, and ammonia concentration, determining an optimum level of and combination of said environmental parameters, for each said cell therapy product within said interior of said fluid container, at each respective said of said time point during said time duration;
at said time points, using electronic data from said electronic data sensors to determine a current level of said environmental parameters of said cell therapy product within said interior of said fluid container;
comparing said current level of said environmental parameters existing within said interior of said fluid container to said optimum level and combination of said environmental parameters associated with said respective time point; and
where said current level of said of said environmental parameters substantially matches said optimum level and combination of environmental parameters, allowing said processing to continue.

18. The system for maintaining production of a cell therapy product in a fluid container of claim 17, additionally comprising:
from said group of environmental parameters determining environmental parameter alterations calculated to adjust said current level of said environmental parameters to match said optimum combinations of environmental parameters; and
where said current level of said environmental parameters varies from said optimum combination of said environmental parameters, initiating environmental parameter alterations determined to adjust said current level of said environmental parameters to match said optimum combinations of environmental parameters.
